(19) United States
(12) Patent Application Publication
(10) Pub. No.: US 2025/0216373 A1
(43) Pub. Date: Jul. 3, 2025

(54) METHOD FOR JUDGING SPATIAL OBLIQUE DISTRIBUTION PATTERNS AND DEEP PROSPECTING AND TARGETING CONCEALED ORE BODIES OF HYDROTHERMAL DEPOSIT CONTROLLED BY STRUCTURES

(71) Applicant: Kunming University of Science and Technology, Kunming City (CN)

(72) Inventors: Runsheng HAN, Kunming City (CN); Yan ZHANG, Kunming City (CN); Jianbiao WU, Kunming City (CN); Qing CHEN, Kunming City (CN); Wenyao LI, Kunming City (CN); Zhonglin GUO, Kunming City (CN); Shengkai WANG, Kunming City (CN); Bangtao SUN, Kunming City (CN); Baosheng HUANG, Kunming City (CN); Ticai HU, Kunming City (CN)

(21) Appl. No.: 18/930,627

(22) Filed: Oct. 29, 2024

(30) Foreign Application Priority Data
Dec. 28, 2023 (CN) 202311830181.6

(51) Int. Cl. G01N 33/24; G01N 3/08

(52) U.S. Cl. CPC G01N 33/24; G01N 3/08; G01N 2203/0019; G01N 2203/0025; G01N 2203/0026; G01N 2203/0262

(57) ABSTRACT

Provided is a method for judging spatial oblique distribution patterns and deep prospecting and targeting concealed ore bodies of a hydrothermal deposit controlled by structures. The method includes: a structural classification ore-controlling law and an combination pattern of ore-controlling structures are determined. A mechanical mechanism of the spatial distribution of ore deposits, ore segments, ore body groups and ore bodies controlled by multi-scale structures is revealed, and an oblique distribution law of ore bodies on a plane and a cross-section is determined. According to the oblique distribution law and the erosion depth of ore body groups and ore segments, pinching-out elevations of the maximum deep extensions of main ore body groups and ore segments are inferred. Deep prospecting and targeting of a concealed ore body is realized, and the deep resource potential is predicted.

### CROSS-REFERENCE TO RELATED APPLICATION

This patent application claims the benefit and priority of Chinese Patent Application No. 202311830181.6 filed with the China National Intellectual Property Administration on Dec. 28, 2023, the disclosure of which is incorporated by reference herein in its entirety as part of the present application.

### TECHNICAL FIELD

The present disclosure relates to method for judging spatial oblique distribution patterns and deep prospecting and targeting concealed ore bodies of a hydrothermal deposit controlled by structures, in particular to a method of exploring and targeting ore deposits, ore segments, ore body groups, ore bodies controlled by metallogenic structures based on different scales (ore deposits, ore segments, ore body groups, ore bodies), which belongs to the field of mineral resources exploration.

### BACKGROUND

The exploration and development of mineral resources is related to the national economy and people's livelihood and the national security. Both in the exploration of non-magmatic hydrothermal deposits and in the magmatic hydrothermal deposits, it is often found that ore bodies not only have the phenomenon of lateral trending, but also the phenomenon of the oblique distribution is the basic feature of a hydrothermal deposit controlled by structures, which is genetically related to a shearing effect. However, up to now, the oblique distribution law of ore bodies is unclear, the formation mechanism of the oblique distribution is unknown, and the oblique distribution law and the mechanical mechanism of ore deposits, ore segments, ore body groups and ore bodies controlled by structures with different scales are rarely studied, which directly restricts the exploration and prospecting breakthrough in the deep and peripheral positioning target areas of the ore deposit. Therefore, the research on the oblique distribution law and the formation mechanism of ore fields, ore deposits, ore segments, ore body groups and ore bodies will undoubtedly play an important guiding role in the prospecting and exploration engineering layout in the deep part and the periphery of the ore deposit and in increasing reserves and increasing production in a new round of prospecting breakthrough strategic actions.

### SUMMARY

The present disclosure provides a method for positioning and predicting deep ore bodies of a hydrothermal deposit by analyzing the oblique distribution law and the mechanical mechanism of ore deposits, ore segments, ore body groups and ore bodies controlled by structures with different scales, that is, (1) based on the fine analysis of metallogenic structures with different scales, and in combination with the spatial distribution characteristics of known ore bodies or mineralized bodies, a structural classification ore-controlling law and combination patterns of ore-controlling structures are determined; (2) based on the structural classification ore-controlling law and the combination patterns of the ore-controlling structures, the oblique distribution laws of long axes of ore body, ore body groups, ore segment and ore deposit on the plane and on the cross-section and the mechanical mechanism of the ore-controlling structures are analyzed and summarized; (3) according to the oblique distribution laws and the erosion depth of ore body group and ore segment, pinching-out elevations of the maximum deep extensions of main ore body group and ore segment are inferred; (4) based on the oblique distribution law of the ore-controlling structures with a plurality of scales on the plane and on the cross-section, the plane occurrence position and the deep vertical occurrence position of a concealed ore bodies in the periphery of the ore deposit are inferred, and in combination with the pinching-out elevations of the maximum deep extensions of ore body group and ore segment, the deep targeting of concealed ore bodies is realized, and the deep resource potential is predicted. Based on this, it can guide the prospecting evaluation and exploration engineering layout in the deep part of the ore deposit, and promote mining enterprises to achieve the goal of increasing reserves and increasing production of resources quickly.

The specific operation of the above method is as follows.

Step 1, analyzing structural classification ore-controlling law and determining combination patterns of ore-controlling structures

Accurately analyzing geometric, kinematic, mechanical and tectonites characteristics of structures with different scales (ore deposit, ore segment, ore body group, ore body) in an ore deposit based on Orefield Geomechanics Theory and Method (Sun Jiacong and Han Runsheng, 2016); screening out metallogenic structures in combination with spatial distribution characteristics (ore-bearing horizon, spatial positioning and occurrence characteristics) of known ore bodies or mineralized bodies; revealing the structural classification ore-controlling law of the ore deposit and determining the combination patterns of the ore-controlling structures.

Step 2, analyzing the mechanical mechanism of the metallogenic structures that controls a spatial oblique distribution of the known ore bodies

Analyzing the kinematic and mechanical characteristics of the metallogenic structures with different scales based on the structural classification ore-controlling law and the combination patterns of the ore-controlling structures, and analyzing controlling multi-scale local stress field characteristics of a single ore body, an ore body group, an ore segment and the ore deposit in combination with the spatial distribution characteristics of the known ore bodies or the mineralized bodies; analyzing and summarizing oblique distribution laws of long axes of the ore bodies, the ore body groups, the ore segments and the ore deposits on a plane and the mechanical mechanism of the ore-controlling structures on the plane; and summarizing the oblique distribution laws of long axes of the ore bodies, the ore body groups, the ore segments and the ore deposits on a cross-section and the mechanical mechanism of the ore-controlling structures on the cross-section based on analysis of metallogenic structures with different dip directions.

The mechanical mechanism of the ore-controlling structures of different scales is as follows:

a long axis of each of the ore deposits: it is controlled by a right-lateral compression-shear surface on the plane and by a compression-shear surface on the cross-section;

a long axis of each of the ore segments: it is controlled by a left-lateral shear-compression surface on the plane and by a shear-compression surface on the cross-section;

a long axis of each of the ore body groups: it is controlled by a left-lateral compression-shear surface on the plane and a compression-shear surface on the cross-section;

a long axis of each of the ore bodies: it is controlled by a right-lateral shear-compression surface on the plane and by a shear-compression surface on the cross-section.

A judgment of the oblique distribution of the ore bodies, the ore body groups, the ore segments and the ore deposits with different scales under the control of different scale structures is as follows:

(1) the ore segments of an ore deposit scale are in a form of a right oblique distribution on the plane, and on the cross-section, if there is a left-lateral shear-compression ore-bearing fracture in a northwest dip direction and in a northeast-southwest strike direction, the ore segments are in a form of a left oblique distribution; if there is a left-lateral shear-compression ore-bearing fracture in a southeast dip direction and in the northeast-southwest strike direction, the ore segments are in a form of a right oblique distribution;

the ore body groups of an ore segment scale are in a form of a left oblique distribution on the plane, and on the cross-section, if there is a left-lateral compression-shear ore-bearing fracture in the northwest dip direction and in the northeast-southwest strike direction, the ore body groups are in a form of a right oblique distribution; if there is a left-lateral compression-shear ore-bearing fracture in the southeast dip direction and in the northeast-southwest strike direction, the ore body groups are in a form of a left oblique distribution;

the ore bodies of an ore body group scale are in a form of a left oblique distribution on the plane, and on the cross-section, if there is a left-lateral shear-compression ore-bearing fracture in the northwest dip direction and in the northeast-southwest strike direction, the ore bodies are in a form of a left oblique distribution; if there is a left-lateral shear-compression ore-bearing fracture in the southeast dip direction and in the northeast-southwest strike direction, the ore bodies are in a form of a right oblique distribution;

(2) the ore segments of the ore deposit scale are in the form of the right oblique distribution on the plane, and on the cross-section, if there is a left-lateral shear-compression ore-bearing fracture in a southwest dip direction and in a northwest-southeast strike direction, the ore segments are in the form of the left oblique distribution; if there is a left-lateral shear-compression ore-bearing fracture in a northeast dip direction and in the northwest-southeast strike direction, the ore segments are in the form of the right oblique distribution;

the ore body groups of the ore segment scale are in the form of the left oblique distribution on the plane, and on the cross-section, if there is a left-lateral compression-shear ore-bearing fracture in the southwest dip direction and in the northwest-southeast strike direction, the ore body groups are in the form of the right oblique distribution; if there is a left-lateral compression-shear ore-bearing fracture in the northeast dip direction and in the northwest-southeast strike direction, the ore body groups are in the form of the left oblique distribution;

the ore bodies of the ore body group scale are in the form of the left oblique distribution on the plane, and on the cross-section, if there is a left-lateral shear-compression ore-bearing fracture in the southwest dip direction and in the northwest-southeast strike direction, the ore bodies are in the form of the left oblique distribution; if there is a left-lateral shear-compression ore-bearing fracture in the northeast dip direction and in the northwest-southeast strike direction, the ore bodies are in the form of the right oblique distribution;

(3) the ore segments of the ore deposit scale are in the form of the right oblique distribution on the plane, and on the cross-section, if there is a left-lateral shear-compression ore-bearing fracture in a north dip direction and in a east-west strike direction, the ore segments are in the form of the left oblique distribution; if there is a left-lateral shear-compression ore-bearing fracture in a south dip direction and in the east-west strike direction, the ore segments are in the form of the right oblique distribution;

the ore body groups of the ore segment scale are in the form of the left oblique distribution on the plane, and on the cross-section, if there is a left-lateral compression-shear ore-bearing fracture in the north dip direction and in the east-west strike direction, the ore body groups are in the form of the right oblique distribution; if there is a left-lateral compression-shear ore-bearing fracture in the south dip direction and in the east-west strike direction, the ore body groups are in the form of the left oblique distribution;

the ore bodies of the ore body group scale are in the form of the left oblique distribution on the plane, and on the cross-section, if there is a left-lateral shear-compression ore-bearing fracture in the north dip direction and in the east-west strike direction, the ore bodies are in the form of the left oblique distribution; if there is a left-lateral shear-compression ore-bearing fracture in the south dip direction and in the east-west strike direction, the ore bodies are in the form of the right oblique distribution;

(4) the ore segments of the ore deposit scale are in the form of the right oblique distribution on the plane, and on the cross-section, if there is a left-lateral shear-compression ore-bearing fracture in a west dip direction and in a south-north strike direction, the ore segments are in the form of the left oblique distribution; if there is a left-lateral shear-compression ore-bearing fracture in a east dip direction and in the south-north strike direction, the ore segments are in the form of the right oblique distribution;

the ore body groups of the ore segment scale are in the form of the left oblique distribution on the plane, and on the cross-section, if there is a left-lateral compression-shear ore-bearing fracture in the west dip direction and in the south-north strike direction, the ore body groups are in the form of the right oblique distribution; if there is a left-lateral compression-shear ore-bearing fracture in the east dip direction and in the south-north strike direction, the ore body groups are in the form of the left oblique distribution;

the ore bodies of the ore body group scale are in the form of the left oblique distribution on the plane, and on the cross-section, if there is a left-lateral shear-compression ore-bearing fracture in the west dip direction and in the south-north strike direction, the ore bodies are in the form of the left oblique distribution; if there is a left-lateral shear-compression ore-bearing fracture in the east dip direction and in the south-north strike direction, the ore bodies are in the form of the right oblique distribution.

Step 3, inferring deep extension of the ore deposit

### 1) Inferring Deep Extension

based on the spatial distribution characteristics of the known ore bodies or the mineralized bodies, further determining spatial distribution characteristics of the ore body group and the ore segment, and then inferring erosion depths of the ore body group and the ore segment.

### 2) Determining the Deep Exploration Orientation

determining pinching-out elevations of maximum deep extensions of the deep part of the ore deposit and the peripheral ore body group or ore segment in the ore deposit according to the oblique distribution laws and the erosion depths of the ore body group and the ore segment.

Step 4, determining positions of concealed ore bodies of the ore deposit and deep prospecting and targeting

based on the oblique distribution laws of the ore body, the ore body group, the ore segment and the ore deposit on the plane and on the cross-section in Step 2, inferring a plane occurrence position and a deep vertical occurrence position of the concealed ore body in a periphery of the ore deposit; and realizing the deep accurate targeting of the concealed ore body in combination with the pinching-out elevations of the maximum deep extensions of the ore body group and the ore segment obtained in Step 3, and inferring the deep resource potential.

Based on the oblique distribution laws of the ore body, ore body group and ore deposit on the plane and on the cross-section, the plane occurrence position of a concealed ore body in the periphery of the mining area and the vertical occurrence position of a concealed ore body in the deep part of the ore deposit are inferred, and in combination with the pinching-out elevations of the maximum deep extensions of ore body group and ore segment obtained in Step (3), the deep targeting is realized, and the deep resource potential is inferred. The present disclosure has at least the following advantages and effects.

(1) The method is suitable for the prospecting prediction of copper, lead, zinc, gold, antimony, silver and other minerals in non-magmatic hydrothermal deposits or magmatic hydrothermal deposits, as well as suitable for the prospecting prediction in polymetallic exploration areas.

(2) The method not only can quickly determine the occurrence positions of deep ore bodies of a hydrothermal deposit which is obviously controlled by structures, and efficiently arrange deep prospecting engineering, but also can infer multi-scale prospecting target areas of ore deposits, ore body groups, ore segments and ore bodies of oblique distribution. In practical application, the prospecting target areas of one or more of ore deposits, ore body groups, ore segments and ore bodies can be inferred according to needs.

(3) The method is simple and easy to apply, which can obviously shorten the exploration period of deep prospecting and reduce the overall cost.

(4) The method can obviously reduce the interference of a single geophysical or geochemical anomaly factor and the influence of multiple solutions.

(5) The method is suitable for the scale of 1:2000 to 1:10000, and the detection depth is no more than 3000 m.

### BRIEF DESCRIPTION OF THE DRAWINGS

FIG. 1A is a stress analysis diagram of the spatial oblique distribution of deep ore body groups in a lead-zinc deposit in the Sichuan-Yunnan-Guizhou metallogenic area in China; FIG. 1B is a vertical projection diagram of the spatial oblique distribution of the deep ore body groups in the lead-zinc deposit in the Sichuan-Yunnan-Guizhou metallogenic area in China;

FIG. 2A is a schematic diagram of a structure with the scale of ore deposit of the lead-zinc deposit in the Sichuan-Yunnan-Guizhou metallogenic area in China; FIG. 2B is a schematic diagram of a structure with the scale of ore segment of the lead-zinc deposit in Sichuan-Yunnan-Guizhou metallogenic area of China; FIG. 2C is a schematic diagram of a structure with the scale of ore body groups of the lead-zinc deposit in Sichuan-Yunnan-Guizhou metallogenic area of China; FIG. 2D is a schematic diagram of a structure with the scale of ore body of the lead-zinc deposit in the Sichuan-Yunnan-Guizhou metallogenic area of China;

FIG. 3A is a plane oblique distribution law diagram of the ore body groups in D ore segment; FIG. 3B is an analysis diagram of a plane mechanical mechanism; FIG. 3C is an oblique distribution law diagram in the cross-section of the ore body group in H ore segment; FIG. 3D is an oblique distribution law diagram in the cross-section of the ore body group in D ore segment; FIG. 3E is a mechanical mechanism analysis diagram in the cross-section of the ore body group in H ore segment; FIG. 3F is a mechanical mechanism analysis diagram in the cross-section of the ore body group in D ore segment;

FIG. 4A shows a diagram of oblique distribution characteristics and a mechanical mechanism of the main ore bodies of the inclined interlayer fracture in the northwest direction (including S, Q and H ore segments); FIG. 4B shows a diagram of oblique distribution characteristics and a mechanical mechanism of the main ore bodies of the inclined interlayer fracture in the southeast direction (including D ore segment), in which 1 represents known ore body, 2 represents predicted ore body, and 3 represents a mineralized alteration variant;

FIG. 5 is an oblique distribution judgment diagram of ore body, ore body group, ore segment and ore deposit on the plane;

FIG. 6 is an oblique distribution law diagram of ore body, ore body group, ore segment and ore deposit controlled by the inclined interlayer fracture in the northwest direction on the cross-section;

FIG. 7 is an oblique distribution law diagram of ore body, ore body group, ore segment and ore deposit controlled by the inclined interlayer fracture in the southeast direction on the cross-section;

FIG. 8 is a schematic diagram of semi-quantitative prediction of the extension depth of each ore segment and each ore body group in a lead-zinc deposit in Sichuan-Yunnan-Guizhou metallogenic area of China; and

FIG. 9 is a plane prediction diagram of the comprehensive TEM and IP geophysical anomalies and the mineralized zone of the lead-zinc deposit, in which 1 represents IP measuring point, 2 represents TEM low resistivity anomaly, 3 represents IP high polarizability anomaly, 4 represents horizontal projection of an ore body estimated by a charging point, 5 represents IP cross-section apparent charging rate anomaly, 6 represents charging point anomaly, 7 represents the estimated extension position of NO. I ore body, 8 represents key prospecting target area, 9 represents projection of the newly discovered ore body, 10 represents predicted mineralized zone, and 11 represents charging measuring point.

### DETAILED DESCRIPTION OF THE EMBODIMENTS

The present disclosure will be further explained in detail by examples, but the scope of protection of the present disclosure is not limited to the described content, and the methods in the examples are all conventional methods unless otherwise specified.

Embodiment 1: oblique distribution law and deep prospecting and targeting of an ore body of a lead-zinc deposit in southwest China

### 1. Study on a Structural Classification Ore-Controlling Law and Determination of an Combination Pattern of Ore-Controlling Structures

Based on Orefield Geomechanics Theory and a method (Sun Jiacong and Han Runsheng, 2016), the special survey of ore-controlling structures is carried out on the surface and deep tunnels in the mining area. The characteristics of geometry, kinematics, mechanics, structural stages and tectonic rocks of fracture structures with different scales are analyzed. The MP left-lateral compression-shear fracture in the northeast direction, the LZH left-lateral shear fracture in the south-north direction, the MMS compound overturned anticline in the northeast direction, the left-lateral torsional fracture in the north-northwestern direction, the tangent layer left-lateral compression-shear fracture in the northeast direction, and the interlayer left-lateral compression-shear fracture in the northeast direction are determined as the metallogenic structures of the ore deposit.

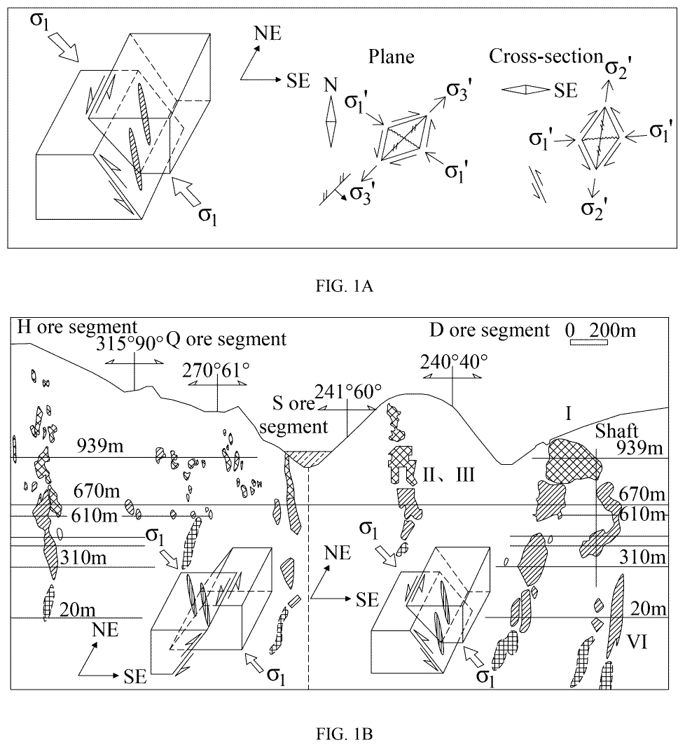

The ore deposit consists of four ore segments, which are bounded by the LZ River running north-south. D ore segment is located in the east, which is divided into NO. I ore body group, NO. II ore body group, NO. III ore body group and NO. VI ore body group. S ore segment, Q ore segment and H ore segment are located in the west area (FIG. 1A and FIG. 1B). The ore bodies are all distributed in the interlayer fracture zone in the northwest wing of MMS compound overturned anticline (FIG. 1A and FIG. 1B). The strata in the east are overturned and tilted in the southeast direction, the middle and shallow part in the strata in the west is tilted in the northwest direction, and the deep part is tilted in the southeast direction. The ore body is lenticular, veined and layered. The ore bodies of No. I ore body group in D ore segment is located in the interlayer fracture zone of D₃zg³⁻² stratum, in which the strike is northeast-southwest, the dip direction is southeast, the dip angle is 60 degrees to 85 degrees, and the southwest is the lateral trending direction. The ore bodies of No. II-III ore body groups are located in the interlayer fracture zone of C₁b and C₂w¹⁻² strata, in which the strike is northeast-southwest, the dip direction is southeast, the dip angle is 60 degrees to 90 degrees, and the southwest is the lateral trending direction. The ore bodies of No. VI ore body group is located in the interlayer fracture zone of D₃zg³⁻¹ stratum, in which the strike is northeast-southwest, the dip direction is southeast, the dip angle is 50 degrees to 70 degrees, and the southwest is the lateral trending direction. The ore bodies of S, Q and H ore segments in the west are located in the interlayer fracture zone of C₂w¹⁻² strata, in which the strike is northeast-southwest, the dip direction is northwest, the dip angle is 70 degrees to 85 degrees, the northeast is the lateral trending direction in the middle and shallow part, and the southwest may be the lateral trending direction in the deep part.

The main ore-bearing fractures, in which the main ore body distributed, of the ore deposit are the interlayer compression-shear fracture zone of the hanging wall anticline of the fracture dominated by the inclined impact-strike slip fault-fold zone in the northeast direction. The mechanical and kinematic mechanism of ore-bearing fractures not only controls the inclined extension of the ore body group more than its strike extension, but also directly controls the characteristics of the extension of the main ore body in the lateral trending direction in the southwest (FIG. 1A and FIG. 1B), and also controls the oblique distribution of ore fields, ore deposits and ore body groups. Combined with the above ore body characteristics, on the basis of studying the main ore-controlling factors (structure and favorable lithologic combination), it is revealed that the fault-fold structure is a typical combination pattern of ore-controlling structures of such deposit. The lead-zinc deposit is the product of the “penetration” of metallogenic fluid driven by the fault-fold structure along the favorable rock combination to form the favorable metallogenic structure.

The structural classification ore-controlling law is as follows: FMB, MP and DB, which are three main fractures in the northeast direction, and their hanging wall derive a series of axial folds in the northeast direction to jointly control the distribution of ore fields. The MP left-lateral compression-shear fracture in the northeast direction and the LZH left-lateral shear fracture in the south-north direction are the primary structures of the ore deposit, which control the spatial distribution of the ore deposit together with the derived MMS compound overturned anticline in the northeast direction which is the secondary structure of the ore deposit. The left-lateral torsional fracture in the north-northwestern direction and the tangent layer left-lateral compression-shear fracture in the northeast direction are the secondary structures of the ore deposit, which control the distribution of ore segments and ore body groups. The “multi-level” interlayer left-lateral compression-shear fracture zone in the northeast direction and the secondary joint fissures are the third and fourth structures of the ore deposit, respectively, which control the shape and the occurrence of a single ore body and the distribution of reticulated and stringer veins.

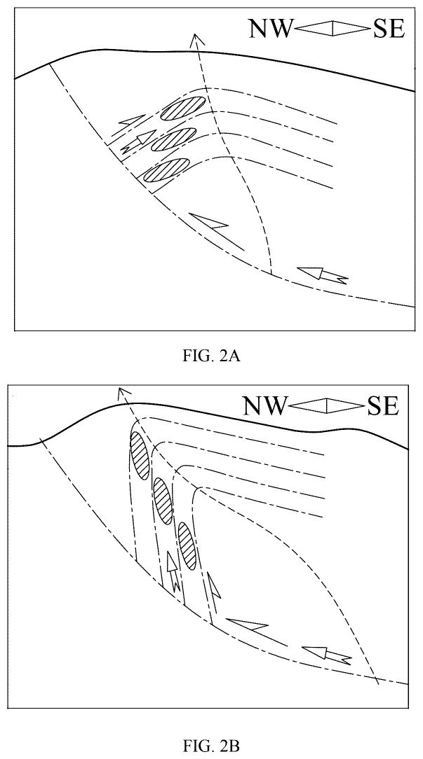

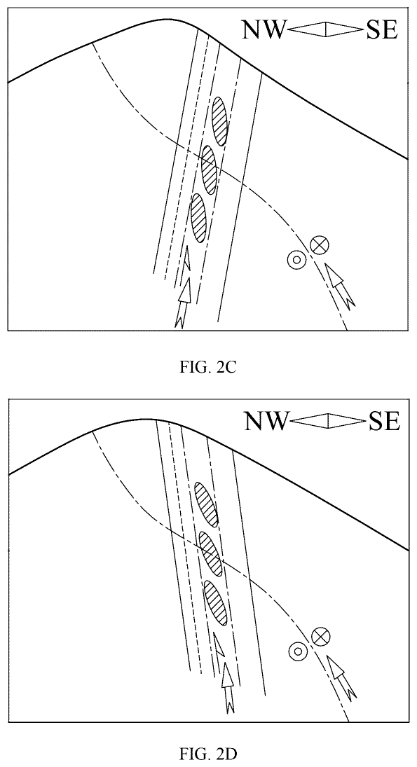

The combination pattern of the ore-controlling structures is as follows: the scale of the ore field, in which the main ore-controlling structure is the combination of an inclined impact-strike slip fault-fold zone and a homoclinal inclined impact-strike slip fault-fold structure; the scale of the ore deposit (the ore segment), in which the main metallogenic structure is fault-fold structure combination, and its combination pattern of the metallogenic structures is reverse fault-anticline combination; the scale of the ore body group (the ore body), in which the main combination patterns of the ore-bearing structures is oblique secondary ore-bearing interlayer fracture-fissure zone (FIGS. 2A-2D).

### 2. Analysis of a Mechanical Mechanism of a Metallogenic Structure That Controls the Spatial Oblique Distribution of Multi-Scale Ore Bodies

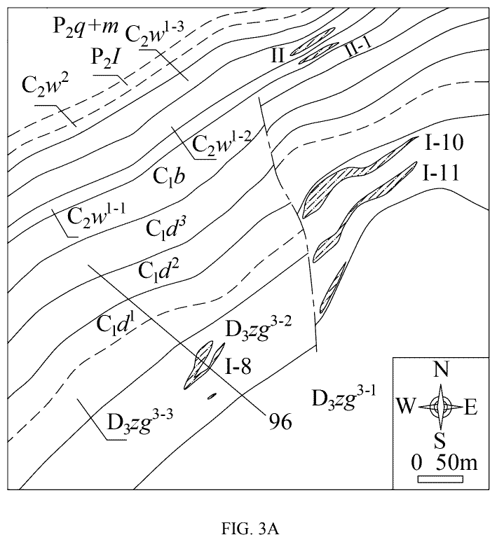

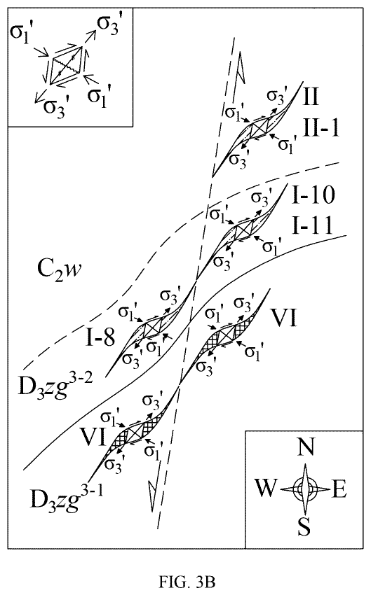

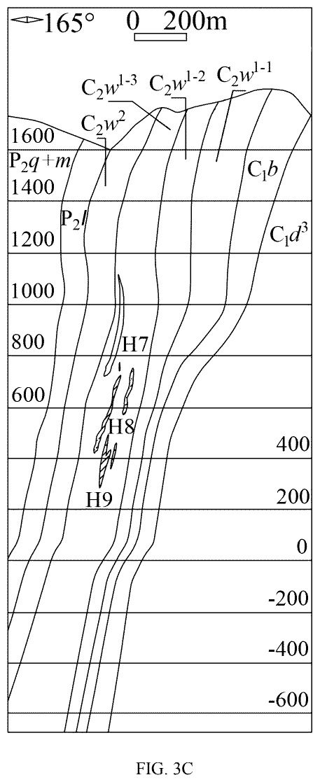

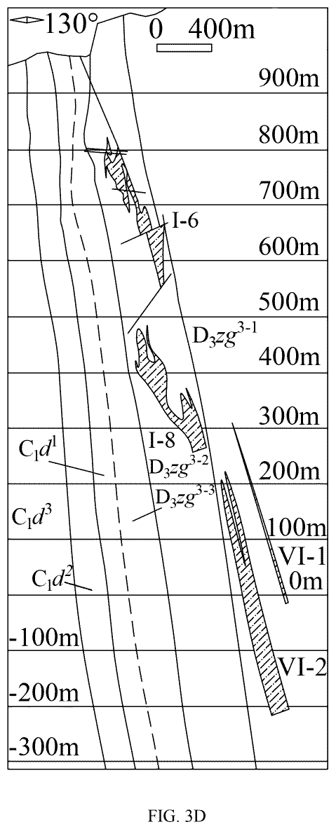

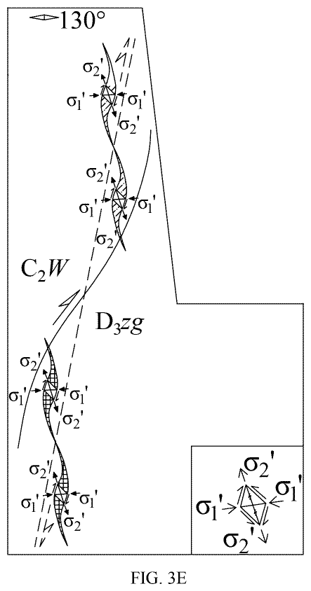

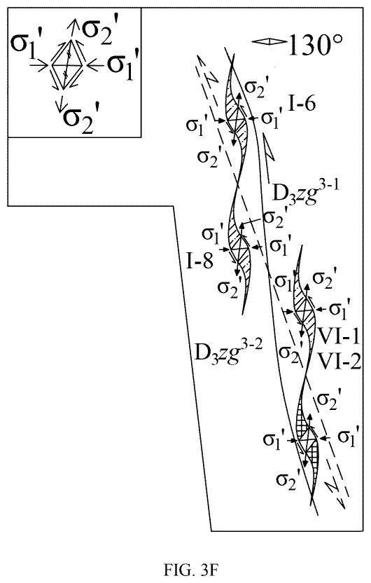

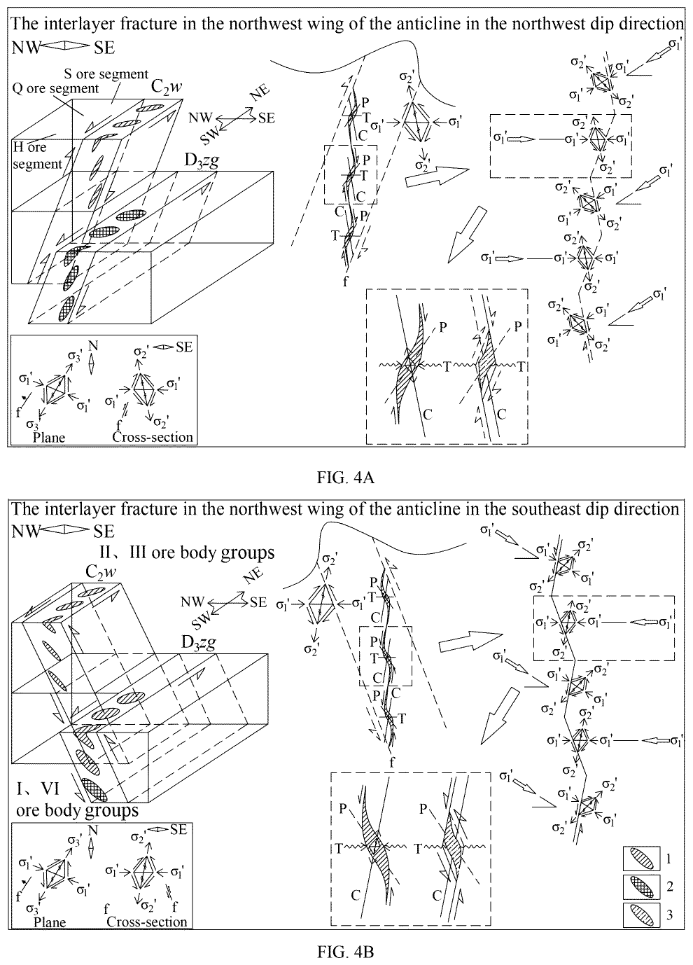

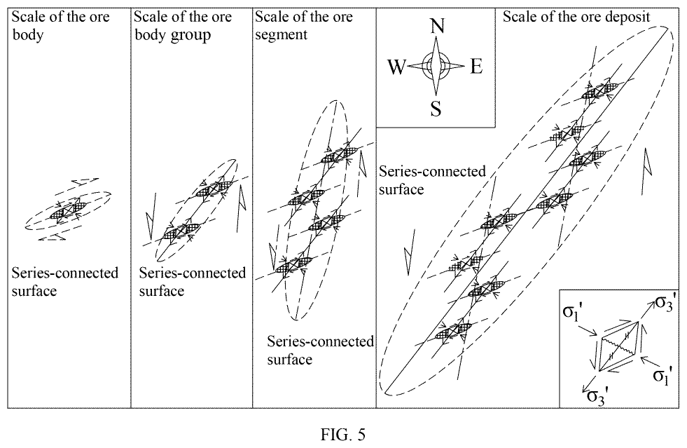

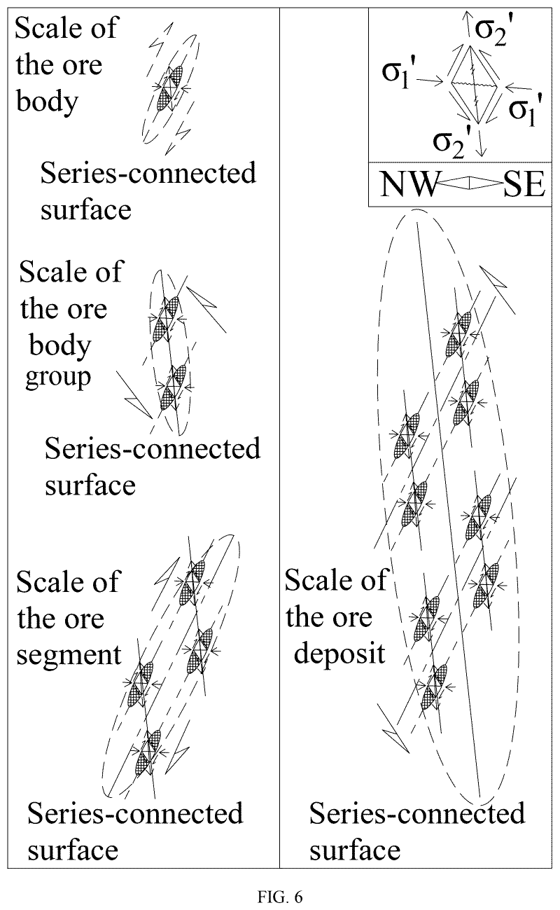

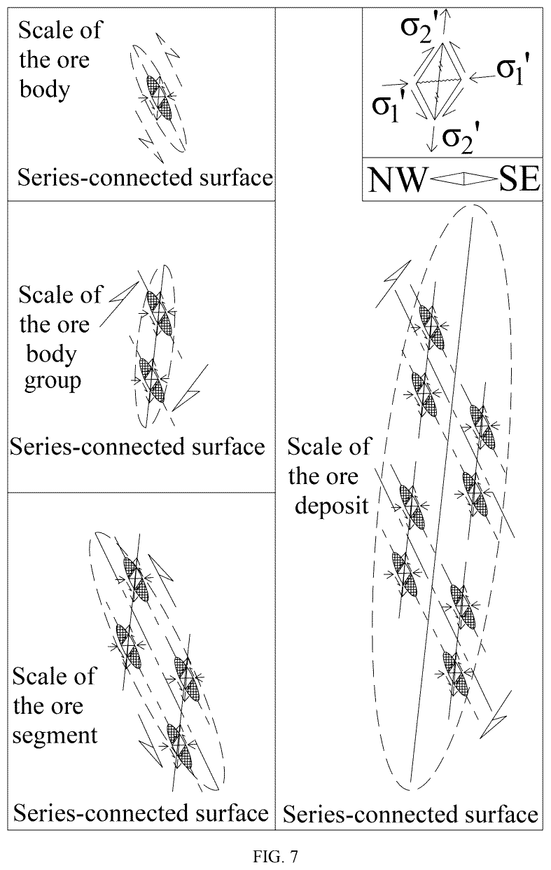

For both the structural classification ore-controlling law and the combination pattern of the ore-controlling structures, there is a sequential genetic relationship between ore-controlling/metallogenic structures with different scales. The spatial distribution of an ore body of a hydrothermal deposit is controlled by the mechanical and kinematic mechanism of ore-bearing fractures during mineralization. The mechanical mechanisms of the formation of ore bodies with different scales are different. Under the same principal compressive stress, the mechanical mechanism of different scales leads to the formation of different oblique distribution laws on the plane and the cross-section. The process of analyzing the mechanical and kinematic characteristics of metallogenic structures with different scales is shown in FIGS. 3A-3F and 4A-4B. FIGS. 5, 6 and 7 show the mechanical mechanism and the oblique distribution law of ore bodies with different scales on the plane and the cross-section.

The lead-zinc deposit is controlled by a fault-fold structure consisting of the MP left-lateral compression-shear fracture in the northeast direction and the MMS compound overturned anticline in the northeast direction, that is, the long axis of the ore deposit. The ore deposit is controlled by the right-lateral compression-shear surface on the plane, and is controlled by the compression-shear surface on the cross-section. The ore deposit at the scale of the ore field is in the form of a right oblique distribution on the plane. On the cross-section, there is a left-lateral compression-shear ore-bearing fracture in the northwest dip direction and in the northeast strike direction, and the ore deposit is in the form of a left oblique distribution; there is a the left-lateral compression-shear ore-bearing fracture in the southeast dip direction and in the northeast strike direction, and the ore deposit is in the form of a right oblique distribution.

Each ore segment is controlled by the left-lateral torsional fracture in the south-north direction and left-lateral torsional fracture in the north-northwestern direction, that is, the long axis of the ore segment. The ore segment is controlled by the left-lateral shear-compression surface on the plane, and is controlled by the shear-compression surface on the cross-section. The ore segment at the scale of the ore deposit is in the form of a right oblique distribution on the plane. On the cross-section, there is a left-lateral shear-compression ore-bearing fracture in the northwest dip direction and in the northeast strike direction, and the ore segment is in the form of a left oblique distribution; there is a the left-lateral shear-compression ore-bearing fracture in the southeast dip direction and in the northeast strike direction, and the ore segment is in the form of a right oblique distribution.

Each ore body group is controlled by the interlayer compression-shear fracture in the northeast direction in the favorable lithologic combination of cover rocks of carbonate rocks and clastic rocks, that is, the long axis of the ore body group. The ore body group is controlled by the left-lateral compression-shear surface on the plane, and is controlled by the compression-shear surface on the cross-section. The ore body group at the scale of the ore segment is in the form of a left oblique distribution on the plane. On the cross-section, there is a left-lateral compression-shear ore-bearing fracture in the northwest dip direction and in the northeast strike direction, and the ore body group is in the form of a right oblique distribution; there is a left-lateral compression-shear ore-bearing fracture in the southeast dip direction and in the northeast strike direction, and the ore body group is in the form of a left oblique distribution.

Each ore body is controlled by the opening space of the compression-shear surface in the interlayer compression-shear fracture zone in the northeast direction, that is, the long axis of the ore body. The ore body is controlled by the right-lateral shear-compression surface on the plane, and is controlled by the shear-compression surface on the cross-section. Therefore, the ore body at the scale of the ore body group is in the form of a left oblique distribution on the plane. On the cross-section, the ore body controlled by the left-lateral shear-compression ore-bearing fracture in the northwest dip direction and in the northeast strike direction is in the form of a left oblique distribution; and the ore body controlled by the left-lateral shear-compression ore-bearing fracture in the southeast dip direction and in the northeast strike direction is in the form of a right oblique distribution.

### 3. Inference of Deep Extension Characteristics of the Ore Deposit and Determine of Prospecting Target Areas

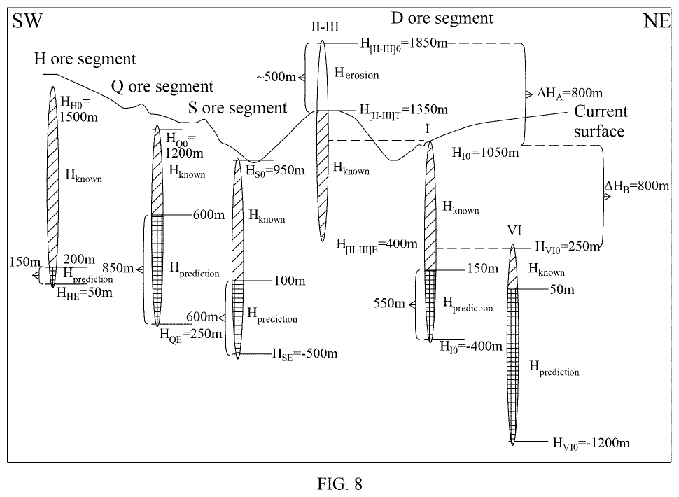

According to the vertical oblique distribution pattern of four ore body groups in D ore segment, H, Q and S ore segments, the extension characteristics of each ore segment and each ore body group and the occurrence positions of new ore body groups and ore bodies are semi-quantitatively predicted (FIG. 8).

On this basis, the erosion depth of ore body groups can be inferred according to the structural ore-controlling laws and the positioning laws of ore body groups (FIG. 8).

1) According to the structural classification ore-controlling law of the ore deposit, the main ore body groups controlled by the fault-fold structure mainly exist in the hanging wall of the main fracture, and the vertical depth is greater than the strike extension, which indicates that the extension depth H_{f} of the main fracture is equivalent to the extension depth Ho of the main ore body:

$${H}_{f} = {H}_{o} \qquad ( 1 )$$

2) According to the distribution law of metallogenic structures at equal intervals, which is consistent with the occurrence law of main ore bodies at equal depths, the main ore bodies have the same vertical distance extension depth. That is, the vertical extension depths of the main ore body groups are:

$${H}_{I} = {H}_{II - III} = {H}_{VI} = {H}_{H} = {H}_{Q} = {H}_{S} \qquad ( 2 )$$

The equal distance of the main ore body groups is: ΔH_{A}=ΔH_{B} (3)

3) It is known that the surface exposed elevation H_{I0} of No. I ore body in No. I mineralized zone is about 1050 m; the exposed elevation H_{VI0} at the beginning of No. VI ore body group is about 250 m; the exposed elevation H_{H0} at the beginning of H ore segment is about 1500 m; the exposed elevation H_{Q0} at the beginning of Q ore segment is about 1200 m; the exposed elevation H_{S0} at the beginning of S ore segment is about 950 m; the maximum exposed elevation of No. II-III ore body groups is about:

$${H}_{( II - III ) T} = 1350 m \qquad ( 4 )$$

4) Because the erosion depth of the surface in the same area is equivalent, in combination with (3), the relative vertical difference of the occurrence depth of the main ore body is:

$$Δ {H}_{A} = Δ {H}_{B} = {H}_{I 0} - {H}_{VI 0} = 1 \; 050 - 250 = 800 m \qquad ( 5 )$$

The vertical erosion depth of the ore body is:

$${H}_{e r o sion} \geq Δ {H}_{A} - ( {H}_{( I I - I I I ) T} - {H}_{I 0} ) = 800 - ( 1 \; 350 - 1050 ) = 500 m \qquad ( 6 )$$

Based on the above research, according to the characteristics of the oblique distribution law of the ore body group and the erosion depth of the ore body group, the pinching-out elevation of the ore body group in the main mineralized zone in the mining area and the maximum depth of further extension are put forward (FIG. 8).

1) According to the original exposed elevation of NO. II-III ore body groups (when the ore deposit is formed):

$${H}_{( I I - I I I ) 0} = 1 \; 350 - 500 = 1850 m \qquad ( 7 )$$

The exposed elevation H_{(II-III)E} of the deep pinching-out end is 400 m to 500 m (calculated by 400 m); therefore, it can be inferred that the initial deep extension elevation difference formed by NO. II-III ore body groups is:

$${H}_{II - III} = {H}_{( I I - I I I ) 0} - {H}_{( I I - 111 ) E} = 1 \; 850 - 400 = 1450 m \qquad ( 8 )$$

Therefore, in combination with (2), the vertical extension depths of the main ore body groups are:

$${H}_{I} = {H}_{VI} = {H}_{H} = {H}_{Q} = {H}_{S} = {H}_{II - III} = 1450 m \qquad ( 9 )$$

2) The pinching-out elevation of the maximum depth extension of No. I ore body group is:

$${H}_{I E} = {H}_{I 0} - {H}_{I} = 1 \; 050 - 1450 = - 400 m \qquad ( 10 )$$

At present, the elevation difference controlled by the exploration engineering is about 850 m to 950 m, and the controlled minimum elevation controlled by the engineering is about 100 m to 200 m. Based on this, it is calculated that the maximum depth of the further extension of the current occurrence elevation of the mineralized zone is 500 m to 600 m, that is:

The controlled minimum elevation (100 m to 200m)−the pinching-out elevation H_{IE}(−400 m)=500 m to 600 m (11)

3) The pinching-out elevation of No. VI ore body group is:

$${H}_{VIE} = {H}_{VI 0} - {H}_{VI} = 250 - 1450 = - 1200 m \qquad ( 12 )$$

At present, the controlled elevation difference is about 150 m to 250 m, and the controlled minimum elevation is 0 m to 100 m. Therefore, it is calculated that the maximum depth of the further extension of the current occurrence elevation in the mineralized zone where the ore body group is located is 1200 m to 1300 m, that is:

The controlled minimum elevation (0 m to 100 m)−the pinching-out elevation

H_{VIE}(−1200 m)=1200 m to 1300 m (13)

4) The pinching-out elevation of the ore body group in H ore segment is:

$${H}_{HE} = {H}_{H 0} - {H}_{H} = 1500 - 1450 = 50 m \qquad ( 14 )$$

At present, the controlled elevation difference of the ore body group is about 1250 m to 1300 m, and the controlled minimum elevation is 200 m to 250 m. Therefore, it is calculated that the maximum depth of the current occurrence elevation in the mineralized zone where the ore body group is located is 150 m to 200 m, that is:

The controlled minimum elevation (200 m to 250 m)−the pinching-out elevation

H_{HE}(50 m)=150 m to 200 m (15)

5) The pinching-out elevation of the maximum depth extension of the ore body group in Q ore segment is:

$${H}_{Q E} = {H}_{Q 0} - {H}_{Q} = 1 \; 200 - 1450 = - 250 m \qquad ( 16 )$$

The controlled elevation difference of the ore body group is about 600 m, and the controlled minimum elevation is about 600 m. Therefore, it is inferred that the maximum depth of the further depth extension of the current occurrence elevation in the mineralized zone where the ore body group is located is about 850 m, that is:

The controlled minimum elevation (600 m)−the pinching-out elevation

H_{QE}(−250 m)=850 m (17)

6) The pinching-out elevation of the maximum depth extension of the ore body group in S ore segment is:

$${H}_{SE} = {H}_{S 0} - H = 950 - 1450 = - 500 m \qquad ( 18 )$$

The controlled elevation difference of the ore body group is about 800 m to 850 m, and the controlled minimum elevation is about 100 m. Therefore, it is inferred that the maximum depth of the further depth extension of the current occurrence elevation of the mineralized zone where the ore body group is located is 600 m to 650 m, that is:

The controlled minimum elevation (100 m)−the pinching-out elevation

H_{SE}(−500 m)=600 m (19)

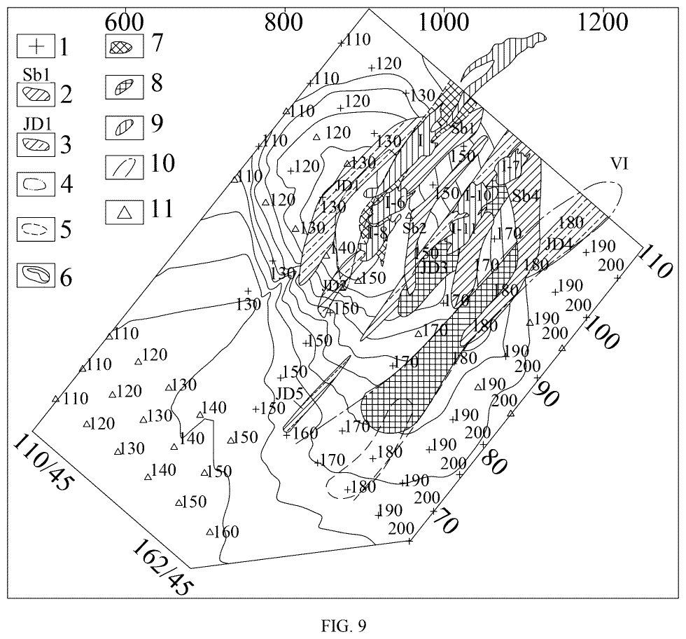

In the prospecting exploration of the ore deposit, for the scale of the ore segment, the deep resource potential of D, Q and S ore segments is great. For the scale of the ore body group, it is necessary to pay attention to the prediction of the position of the left-oblique distribution ore body group in the H, Q and S ore segments located on the plane in the D₃zg (FIGS. 4A-4B). According to the left-oblique distribution law of ore body groups on the plane, it can be predicted that there are left-oblique distribution ore body groups on one side of ore body groups such as I, I-6 and I-8, and ore body groups such as I-7, I-10 and I-11 in D ore segment. The prediction is consistent with the inference of the geophysical anomaly zone (FIG. 9). The engineering verification shows that there is NO. VI ore body group. For the scale of the ore body, according to the oblique distribution law of ore bodies on the plane and on the cross-section, new ore body occurrence positions are predicted. The predicted ore body positions in No. I and No. VI ore body groups in the D ore segment are focused, which are in the form of a right oblique distribution with the deep part of the known ore bodies (such as I-8, I-11, VI-1, VI-2, etc.) (FIGS. 3A-3F). At present, some target areas have been verified. At the same time, the resource potential of the ore body group of the deep part can be inferred according to the predicted occurrence elevation of the ore body group and the average grade and scale of ores in each ore segment, which provides a basis for the decision-making of increasing reserves and increasing production of mining enterprises and for deep exploration work. The example shows that the method is feasible.

What is claimed is:

1. A method for judging spatial oblique distribution patterns and deep prospecting and targeting concealed ore bodies of a hydrothermal deposit controlled by structures, comprising the following steps:
(1) analyzing a structural classification ore-controlling law and determining an combination pattern of ore-controlling structures
analyzing geometric, kinematic, mechanical and tectonite characteristics of structures with different scales in an ore deposit based on an ore field geomechanics theory and a method thereof; screening out metallogenic structures in combination with spatial distribution characteristics of known ore bodies or mineralized bodies; revealing the structural classification ore-controlling law of the ore deposit and determining the combination pattern of the ore-controlling structures; wherein the spatial distribution characteristics include ore-bearing horizon, spatial positioning and occurrence characteristics;
(2) analyzing a mechanical mechanism of the metallogenic structures that controls a spatial oblique distribution of the known ore bodies
analyzing the kinematic and mechanical characteristics of the metallogenic structures with different scales based on the structural classification ore-controlling law and the combination pattern of the ore-controlling structures, and analyzing and controlling local stress field characteristics of a single ore body, an ore body group, an ore segment and the ore deposit in combination with the spatial distribution characteristics of the known ore bodies or the mineralized bodies; analyzing and summarizing oblique distribution laws of long axes of ore bodies, ore body groups, ore segments and ore deposits on a plane and the mechanical mechanism of the ore-controlling structures on the plane; and revealing the oblique distribution laws of long axes of the ore bodies, the ore body groups, the ore segments and the ore deposits on a cross-section and the mechanical mechanism of the ore-controlling structures on the cross-section based on analysis of metallogenic structures with different dip directions;
(3) inferring deep extension of the ore deposit
based on the spatial distribution characteristics of the known ore bodies or the mineralized bodies, further determining spatial distribution characteristics of the ore body groups and the ore segments, and then inferring erosion depths of the ore body groups and the ore segments; and determining pinching-out elevations of maximum deep extensions of the ore body groups and the ore segments in the ore deposit according to the oblique distribution laws and the erosion depths of the ore body groups and the ore segments;
(4) determining concealed ore bodies position of the ore deposit and deep prospecting and targeting
based on the oblique distribution laws of the ore bodies, the ore body groups, the ore segments and the ore deposits on the plane and on the cross-section in Step (2), inferring a plane occurrence position and a deep vertical occurrence position of the concealed ore bodies in a periphery of the ore deposit; and realizing the deep accurate targeting of the concealed ore bodies in combination with the pinching-out elevations of the maximum deep extensions of the ore body groups and the ore segments obtained in Step (3);
carrying out a exploration engineering layout based on the plane occurrence position and the deep vertical occurrence position of the concealed ore body and the deep accurate targeting of the concealed ore body.

2. The method for judging spatial oblique distribution patterns and deep prospecting and targeting the concealed ore bodies of the hydrothermal deposit controlled by structures according to claim 1, wherein the different scales refer to ore deposits, ore segments, ore body groups and ore bodies.

3. The method for judging spatial oblique distribution patterns and deep prospecting and targeting the concealed ore bodies of the hydrothermal deposit controlled by structures according to claim 1, wherein the mechanical mechanism of the ore-controlling structures with different scales in Step (2) is as follows:
a long axis of each of the ore deposits: it is controlled by a right-lateral compression-shear surface on the plane and by a compression-shear surface on the cross-section;
a long axis of each of the ore segments: it is controlled by a left-lateral shear-compression surface on the plane and by a shear-compression surface on the cross-section;
a long axis of each of the ore body groups: it is controlled by a left-lateral compression-shear surface on the plane and a compression-shear surface on the cross-section;
a long axis of each of the ore bodies: it is controlled by a right-lateral shear-compression surface on the plane and by a shear-compression surface on the cross-section.

4. The method for judging spatial oblique distribution patterns and deep prospecting and targeting the concealed ore bodies of the hydrothermal deposit controlled by structures according to claim 3, wherein a judgment of the oblique distribution of the ore bodies, the ore body groups, the ore segments and the ore deposits in Step (2) is as follows:
(1) the ore segments of an ore deposit scale are in a form of a right oblique distribution on the plane, and on the cross-section, if there is a left-lateral shear-compression ore-bearing fracture in a northwest dip direction and in a northeast-southwest strike direction, the ore segments are in a form of a left oblique distribution; if there is a left-lateral shear-compression ore-bearing fracture in a southeast dip direction and in the northeast-southwest strike direction, the ore segments are in a form of a right oblique distribution;
the ore body groups of an ore segment scale are in a form of a left oblique distribution on the plane, and on the cross-section, if there is a left-lateral compression-shear ore-bearing fracture in the northwest dip direction and in the northeast-southwest strike direction, the ore body groups are in a form of a right oblique distribution; if there is a left-lateral compression-shear ore-bearing fracture in the southeast dip direction and in the northeast-southwest strike direction, the ore body groups are in a form of a left oblique distribution;
the ore bodies of an ore body group scale are in a form of a left oblique distribution on the plane, and on the cross-section, if there is a left-lateral shear-compression ore-bearing fracture in the northwest dip direction and in the northeast-southwest strike direction, the ore bodies are in a form of a left oblique distribution; if there is a left-lateral shear-compression ore-bearing fracture in the southeast dip direction and in the northeast-southwest strike direction, the ore bodies are in a form of a right oblique distribution;
(2) the ore segments of the ore deposit scale are in the form of the right oblique distribution on the plane, and on the cross-section, if there is a left-lateral shear-compression ore-bearing fracture in a southwest dip direction and in a northwest-southeast strike direction, the ore segments are in the form of the left oblique distribution; if there is a left-lateral shear-compression ore-bearing fracture in a northeast dip direction and in the northwest-southeast strike direction, the ore segments are in the form of the right oblique distribution;
the ore body groups of the ore segment scale are in the form of the left oblique distribution on the plane, and on the cross-section, if there is a left-lateral compression-shear ore-bearing fracture in the southwest dip direction and in the northwest-southeast strike direction, the ore body groups are in the form of the right oblique distribution; if there is a left-lateral compression-shear ore-bearing fracture in the northeast dip direction and in the northwest-southeast strike direction, the ore body groups are in the form of the left oblique distribution;
the ore bodies of the ore body group scale are in the form of the left oblique distribution on the plane, and on the cross-section, if there is a left-lateral shear-compression ore-bearing fracture in the southwest dip direction and in the northwest-southeast strike direction, the ore bodies are in the form of the left oblique distribution; if there is a left-lateral shear-compression ore-bearing fracture in the northeast dip direction and in the northwest-southeast strike direction, the ore bodies are in the form of the right oblique distribution;
(3) the ore segments of the ore deposit scale are in the form of the right oblique distribution on the plane, and on the cross-section, if there is a left-lateral shear-compression ore-bearing fracture in a north dip direction and in a east-west strike direction, the ore segments are in the form of the left oblique distribution; if there is a left-lateral shear-compression ore-bearing fracture in a south dip direction and in the east-west strike direction, the ore segments are in the form of the right oblique distribution;
the ore body groups of the ore segment scale are in the form of the left oblique distribution on the plane, and on the cross-section, if there is a left-lateral compression-shear ore-bearing fracture in the north dip direction and in the east-west strike direction, the ore body groups are in the form of the right oblique distribution; if there is a left-lateral compression-shear ore-bearing fracture in the south dip direction and in the east-west strike direction, the ore body groups are in the form of the left oblique distribution;
the ore bodies of the ore body group scale are in the form of the left oblique distribution on the plane, and on the cross-section, if there is a left-lateral shear-compression ore-bearing fracture in the north dip direction and in the east-west strike direction, the ore bodies are in the form of the left oblique distribution; if there is a left-lateral shear-compression ore-bearing fracture in the south dip direction and in the east-west strike direction, the ore bodies are in the form of the right oblique distribution;
(4) the ore segments of the ore deposit scale are in the form of the right oblique distribution on the plane, and on the cross-section, if there is a left-lateral shear-compression ore-bearing fracture in a west dip direction and in a south-north strike direction, the ore segments are in the form of the left oblique distribution; if there is a left-lateral shear-compression ore-bearing fracture in a east dip direction and in the south-north strike direction, the ore segments are in the form of the right oblique distribution;
the ore body groups of the ore segment scale are in the form of the left oblique distribution on the plane, and on the cross-section, if there is a left-lateral compression-shear ore-bearing fracture in the west dip direction and in the south-north strike direction, the ore body groups are in the form of the right oblique distribution; if there is a left-lateral compression-shear ore-bearing fracture in the east dip direction and in the south-north strike direction, the ore body groups are in the form of the left oblique distribution;
the ore bodies of the ore body group scale are in the form of the left oblique distribution on the plane, and on the cross-section, if there is a left-lateral shear-compression ore-bearing fracture in the west dip direction and in the south-north strike direction, the ore bodies are in the form of the left oblique distribution; if there is a left-lateral shear-compression ore-bearing fracture in the east dip direction and in the south-north strike direction, the ore bodies are in the form of the right oblique distribution.
